(19) United States
(12) Patent Application Publication
(10) Pub. No.: US 2007/0207482 A1
(43) Pub. Date: Sep. 6, 2007

(54) WOBBLE SEQUENCING

(75) Inventors: George M. Church, Brookline, MA (US); Jay Shendure, Chagrin Falls, OH (US); Gregory J. Porreca, Ocean City, NJ (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/670,588

(22) Filed: Feb. 2, 2007

Related U.S. Application Data
(63) Continuation of application No. PCT/US05/27695, filed on Aug. 4, 2005.
(60) Provisional application No. 60/598,610, filed on Aug. 4, 2004.
(60) Provisional application No. 60/692,718, filed on Jun. 22, 2005.

(51) Int. Cl. C12Q 1/68; C07H 21/04

(52) U.S. Cl. 435/6; 536/25.32

(57) ABSTRACT

Novel methods and compositions for DNA sequencing are provided. The methods described herein are useful for sequencing homopolymeric regions of DNA. The methods also prevent the accumulation of mistakes and inefficiencies in the sequencing reaction.

### CROSS REFERENCE OF RELATED APPLICATIONS

This application is a continuation of PCT application no. PCT/US2005/027695, designating the United States and filed Aug. 4, 2005; which claims the benefit of the filing date of U.S. provisional application No. 60/598,610, filed Aug. 4, 2004; and U.S. provisional No. 60/692,718, filed Jun. 22, 2005; all which are hereby incorporated herein by reference in their entirety.

### STATEMENT OF GOVERNMENT INTERESTS

This invention was made with Government support under Award Numbers IP50 HG003170, awarded by the Centers of Excellence in Genomic Science (CEGS); and DE-FG02-02ER63445, awarded by Genomes to Life (GTL). The Government has certain rights in the invention.

### FIELD OF THE INVENTION

The present invention relates to novel methods and compositions for DNA sequencing. The methods described herein are useful for sequencing homopolymeric regions of DNA.

### BACKGROUND OF THE INVENTION

Current state-of-the-art in sequencing-by-synthesis relies on a single sequencing primer, with a known sequence, followed by cyclic additions of a single nucleotide species at each cycle and detection of incorporation events (e.g., C-A-G-T-C-A-G-T . . . ) via fluorescence or light. Examples of these methods include fluorescent in situ sequencing (FISSEQ) and pyrosequencing. A major problem for both of these approaches is that it is very difficult to decode consecutive runs of the same base in the unknown sequence (i.e., hompolymeric runs), and it is difficult to distinguish single from multiple incorporation events. As approximately 44% of nucleotides are part of a homopolymeric run, this is obviously a major consideration. Most efforts to circumvent this problem involve the development of reversibly terminating nucleotides, which cause a variety of difficulties.

A second problem with the FISSEQ approach is that the set of polymerases typically utilized in such reactions do not efficiently incorporate nucleotides due to the high density of modified nucleotides. For that reason, a large fraction of unlabeled nucleotides are introduced, thus reducing the overall density of modification and extending read-lengths. This results in less labeled nucleotide and, accordingly, less signal. Accordingly, the present invention is directed to novel methods of sequencing that circumvent these problems and provides advantages over methods of sequencing known in the art.

### SUMMARY

The present invention provides novel sequencing methods designed to circumvent problems associated with sequencing-by-synthesis methods known in the art. Although the methods described herein are based on sequencing by polymerase-extension, they differ from FISSEQ and pyrosequencing in that base-additions are not “progressive.” Instead, after a given single-base-extension (SBE), the sequencing primer is stripped from the bead-immobilized templates and a new primer is hybridized. Thus to get beyond the first base, each sequencing primer in the set “reaches” out to a defined position in the unknown unique sequence of the template (e.g., to the fourth base or the fifth base). A sequencing primer, from 5′ to 3′, thus consists of an “anchor sequence” that is complementary to the constant sequence on the template, and a defined number of additional bases (e.g., universal, degenerate and/or natural bases), that will hybridize to the unknown sequence regardless of what it is. If, for example, there are three fixed universal bases, then the sequencing primer is positioned to sequence the fourth base via SBE with labeled nucleotides. After a single-base-extension and data acquisition, extended and unextended primers are stripped (e.g., with heat) and a new primer is annealed that has a different number of universal bases, thus querying a different base-position within the unknown sequence. Thus in this simplest iteration of the scheme, one only needs a set of N primers to achieve a read-length of N.

The present invention provides many advantages over sequencing methods known in the art. The methods described herein: 1) provide a quick solution to the problem of sequencing homopolymers; 2) enable manual mistakes and biochemical inefficiencies to be non-cumulative; 3) greatly expedite the technology development for longer reads (i.e. don't have to cycle out to test a method for improving read-lengths); 4) provide better signals than are obtained by the FISSEQ system currently used in the art (i.e., in which a desire for signal has to be balanced against a desire to minimize the fraction of extended templates with cleaved linker as it inhibits the polymerase); and 5) greatly increase the choice and amounts of enzyme (polymerase or ligase) due to the lack of a requirement to take extensions to completion.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 depicts primer information. The first column of numbers indicates the cycle number assigned to a given query. The second and third columns indicate the sequencing primer used, and the fourth column indicates the conditions of hybridization. The fifth column indicates the base(s) used to extend, and the 6^{th} column indicates the templates expected to add. The remaining columns indicate the best-fit slope coefficient for adders and non-adders, and finally the ratio of these values. TR=Texas Red.

FIG. 2 depicts an extension with 37 C.8N.CG, sequencing bases 10, 11, 12 on T4. Blue indicates bases that were sequenced; yellow indicates bases attempted and failed; uncolored indicates bases that were not attempted.

FIG. 3 depicts sequencing on emulsion beads.

FIG. 4 depicts primer information for primers that extended either T2, T3 or T4.

FIG. 5 depicts bases that were sequenced. Blue indicates bases that were sequenced; yellow indicates bases attempted and failed; uncolored indicates bases that were not attempted.

FIG. 6 depicts sequencing on emulsion beads.

FIG. 7 is a schematic depicting query of tag positions (-5) by mismatch ligation.

FIGS. 8A and 8B is a schematic depicting unique tags and queries that will ligate.

FIGS. 9A and 9B is a schematic of the method of the present invention.

FIG. 10 is a four color depiction of four possible base calls.

FIGS. 11 is a graph showing variation in accuracy over each of 26 cycles of non-progressive sequencing.

### DETAILED DESCRIPTION

In the methods described herein, DNA sequences of numerous features are obtained in parallel by cycles of hybridization of sequencing primers that contain universal, degenerate, and/or specific bases at positions of unknown sequence, followed by single-base-extension with polymerase and nucleotide. As polymerases generally only extend from terminally-matched nucleotides, when an extension occurs, the identity of the bases complementary to specific bases present at the 3′ terminus of a given sequencing primer is revealed. Furthermore, use of modified nucleotides with different fluorescent labels reveals the identity of the incorporated nucleotide. As a given sequencing primer is designed with a known number of universal or degenerate nucleotides, and a known number of specific nucleotides, one knows the specific position within the unknown template that one is sequencing.

The methods of the invention include the use of “degenerate bases” which are intended to include, but are not limited to, primer mixes that contain all possible sequences at unknown positions. The methods of the invention also include the use of universal bases at some or all of the primer positions. “Universal bases” are intended to include, but are not limited to, synthetic nucleotide analogs that ideally pair with equal affinities to each of the natural nucleotides, and are readily accepted as substrates by natural enzymes. Examples of universal bases include 5-nitroindole, 3-nitropyrole, deoxyinosine, and the like. The methods of the invention further include the use of natural bases, wherein sequencing primer oligonucleotides are synthesized with fully degenerate positions, such that all possible sequencing primers (or some random subset of all possibilities) are present during hybridization. Without intending to be bound by theory, overall efficiency could be improved by enzyme engineering for greater permissiveness with respect to mismatches (e.g., the M1/M4 variants of Taq) or alterations to the primer design strategy.

In one embodiment, methods of the invention are directed to fixing the terminal two bases of a given sequencing primer, but allowing the remainder of bases at “universal” positions to be synthesized with fully-degenerate natural bases. The disadvantage of this compromise is that 16 separate hybridizations are required for each “reach” length (4² combinations of the two terminal bases). This is mitigated by the fact that polymerases don't extend off of mispaired termini very well, so a given extension set reveals the identity of both the two terminal bases and the extended base. So the average efficiency of the process is 3/16=0.188 bases per cycle.

Non-terminator FISSEQ, by comparison, yields approximately 0.50 bases-per-cycle (assuming no homopolymer resolution and thus counting multi-base runs as single extensions). By this consideration, achieving an identical read-length would require approximately 2.67 times as many cycles in the 2 bp-matched-wobble-sequencing system.

This invention is further illustrated by the following examples, which should not be construed as limiting. The contents of all references, patents and published patent applications cited throughout this application are hereby incorporated by reference in their entirety for all purposes.

### EXAMPLE I

### Cycle Protocol

Typical cycles were as follows:

1. Hybridize sequencing primer (15 minutes, 10 μM primer in 6×SSPE, 40-50° C.)

2. Extend (4 minutes, SSB+polymerase+nucleotide)

3. Wash (2 minute)

4. Image acquisition

5. Strip primer (5 minutes, Wash 1E, 70′ C)

If the wobble-bases were fixed (poly-A, poly-G, poly-C, or poly-T instead of poly-N), extensions were no longer efficient. Without intending to be bound by theory, this indicates that some degree of “sorting” is going on during the hybridization that is critical to the overall process working. Hoping for this to occur, the “anchor sequence” is purposefully short (Tm=37° C. if it were alone), weighting the hybridization process to depend to a greater degree on the “wobble” or degenerate sequences. Initial data indicated that SEQUENASE™ was significantly better than Klenow for this approach. Primer-stripping was initially very inefficient with beads. It only started working when the bead array was fabricated such that the beads were embedded in the gel near the gel-liquid interface (opposite the glass surface, or “top-layered”).

### EXAMPLE II

### Primer Nomenclature

A typical primer-name below is “37C.2N.CA”. For the primers described herein, the anchor sequence is a trimmed version of the original FISSEQ primer for the T1 . . . T5 template. The “37C” (or “23C” or the like) indicates the extent to which it has been trimmed (i.e. 37C is the Tm of the anchor sequence if it were a stand-alone primer). The “2N” indicates that the anchor-sequence is followed by two full “wobble” or degenerate bases, and the CA indicates the fixed two terminal bases. This primer would extend to the 5th base, thus sequencing 3 bases (base 3, 4 and 5) on 1/16^{th} of the templates of a random library.

In the examples below, primers with even numbers of “wobble” or degenerate bases and terminal bases that match at least one of the five T1 . . . T5 templates were focused on to ensure extension at every cycle. For a given “reach-length,” this was approximately ₁/₄th of the primers that would be required in a real sequencing experiment involving sequencing of genomic fragments. However, this estimate is slightly conservative in that one could do multiples of three for the number of “wobble” or degenerate bases, rather than multiples of two. Some optional redundancy was built in. For example, 37 C.2N.XX sequences bases 3, 4 and 5. 37 C.4N.XX sequences bases 5, 6 and 7. Thus, base 5 was sequenced twice (as is base 7, base 9, etc.)

### EXAMPLE III

### Proof of Principle on Loaded Beads

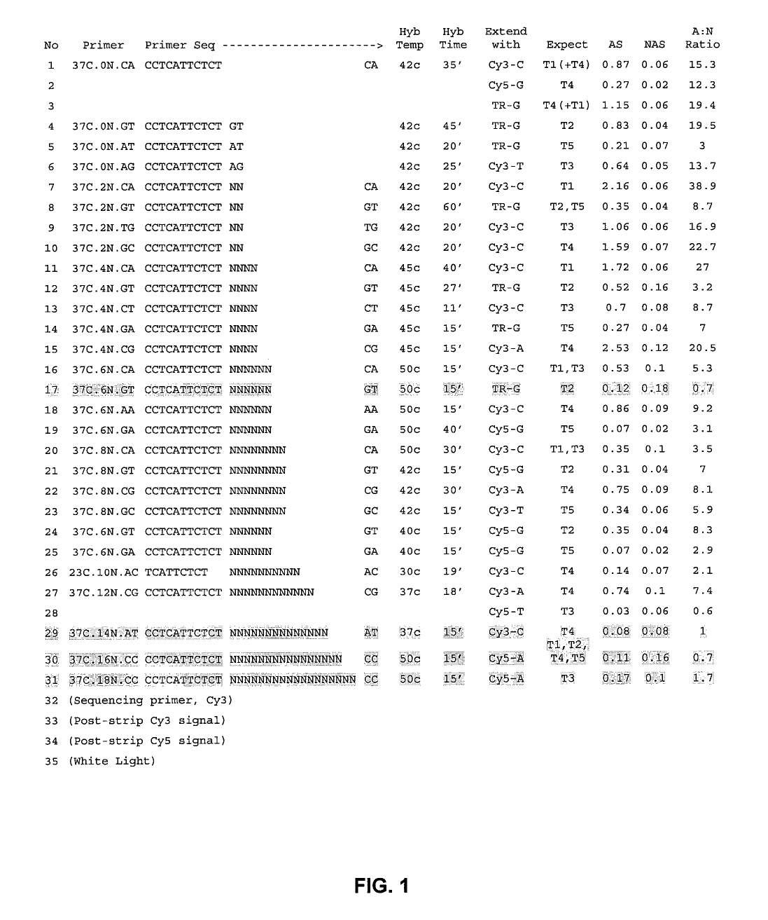

FIG. 1 depicts results from top-layered, 1 μM beads with loaded T1. . . T5 templates. These are primers that would be required in a full sequencing experiment on unknown sequence. Primers were ordered to sequence through to the 11^{th} base on all five templates (37 C.0N.XX through 37 C.8N.XX). Only one primer was ordered for 37 C.10N.XX through 37 C.18N.XX.

Failures are listed in yellow. Without intending to be bound by theory, the first failure (cycle 17), was likely due to manual error in preparing the extension reagent mix, as its repeat (cycle 24) was successful, and this primer worked well in the emulsion-bead experiment below. Without intending to be bound by theory, the remaining failures correlate with attempts at longer reads. The 37 C.12N.CG primer, interestingly, works quite well for one template but not another. In a subsequent experiment, using SEQUENASE™ instead of Klenow resulted in both templates working with this primer. SEQUENASE™ also yields greater signal in general than Klenow in this protocol.

Without intending to be bound by theory, several trends emerged: a) there was poor performance of “G” extensions, which was improved using SEQUENASE™; and b) poor performance of the T5 template in terms of signal yield at any given cycle when it was expected to extend. This outcome may be explained by the shortening of the anchor of the sequencing primer.

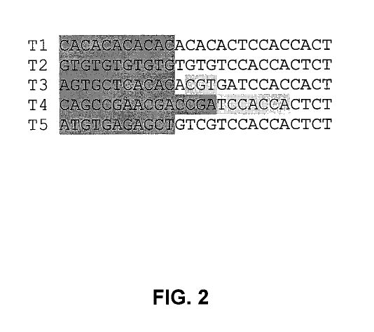

Approximately 11-base-pair reads were obtained from all five templates, and all observations appear consistent. A 15-bp read was obtained on one of the templates (T4), but results were not consistent (i.e. cycle 28) and failure was experienced beyond base 15 (cycles 29-31). Extension was performed with 37 C.8N.CG, sequencing bases 10,11,12 on T4 (FIG. 2).

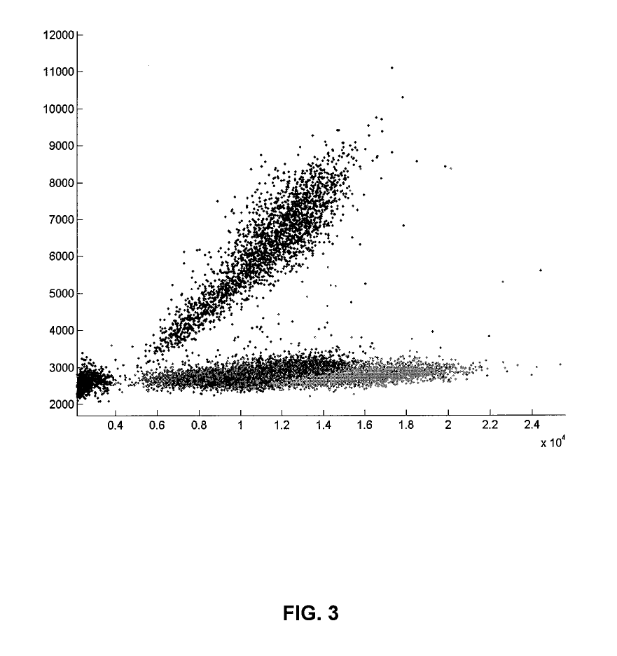

Since the above worked so well, the experiment was repeated on emulsion-generated beads top-layered (FIG. 3). The templates were diluted independently, only mixing them as they went into the emulsion mix. The reason for this is that they are single-stranded, and this procedure minimizes their binding to one another, which confounds results. However, the ratios of the five templates deviated from 1:1. The initial set of primers used on these templates were the 37 C.0N.XX series, which essentially establishes the identity of each bead. As the fraction of beads with 1 or more template was high, it was not surprising that a high fraction of non-clonal beads was observed. Only approximately 1% of the gel (25 frames) was imaged at each cycle. The overall numbers were as follows: no template, 29,658; weakly amplified, 10,164; strong clonal, 13,350; T1=57; T2=8,945; T3=2,165; T4=1,834; T5=349; strong non-clonal, 7,668; and total, 60,840.

The numbers are generally consistent with what one would expect from Poisson statistics, but with a modest excess of non-clonal beads. Without intending to be bound by theory, these data indicate that some fraction of the “no template” beads actually don't participate in the distribution (e.g., they are excluded because they are in the oil compartment, or in a compartment that is too small to initiate PCR and the like).

### EXAMPLE IV

Primers That Extended Either T2, T3, or T4

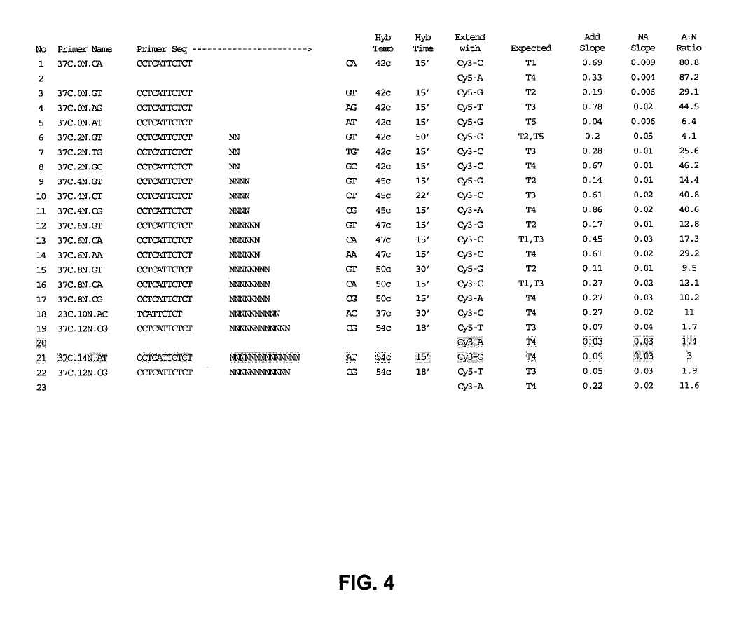

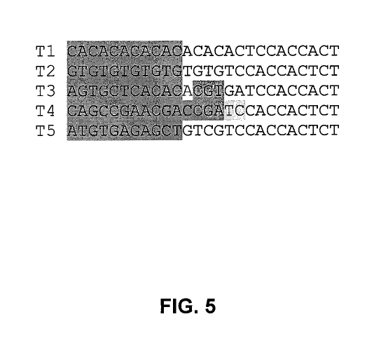

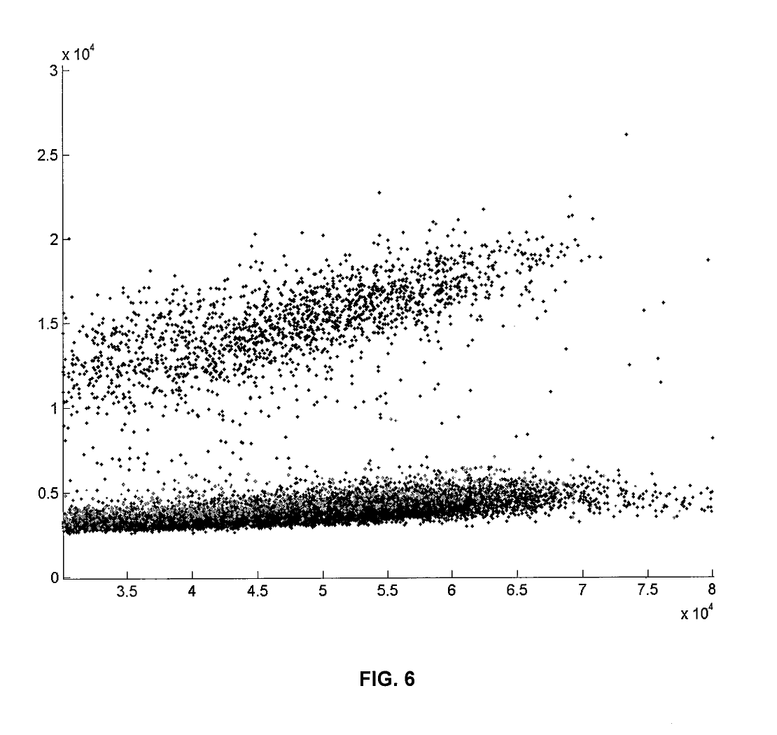

The initial analysis of clonality and identity, which were based on the 37 C.0N.XX primers, led to the focus on primers that extended either T2, T3, or T4, as these dominated the slide (FIGS. 4 and 6). Relative to the above there are also changes to the hybridization conditions and modified nucleotides, but the most important difference (other than the fact that these are emulsion-generated beads) was that SEQUENASE™ was utilized instead of Klenow. Extension was performed with 37 C.8N.CG, sequencing bases 10, 11, 12 on T4 using emulsion-beads instead of loaded beads (FIG. 5).

On cycle 19/20 (FIG. 4), stripping was performed before reading the Cy3 signal out. Interestingly, less than 30 seconds in Wash IE at 70° C. was sufficient for stripping, or at least for redistribution of signal amongst the beads. Thus, cycles 22 and 23 were repeated with 37 C.12N.CG.

What worked and what didn't work was based on visual inspection of the graphs. Thus, without intending to be bound by theory, even though 37.12N.CGiT had lower “ratios” than 37 C.14N.ATiC, it still appears to have worked, whereas 37 C.14N.ATiC appeared not to have worked.

The slide was stripped and sequencing primer was re-annealed at the conclusion to determine to what extent the templates had fallen off due to heat exposure and the like. The difference between the two sets of images (pre-sequencing and post-sequencing) was negligible. The two sets of images were strikingly consistent with one another, which indicated that template was not being lost over the course of the experiment. This inspection also demonstrated quite clearly that the extent of gel warping over the approximately 20 cycles was negligible. Good signal was obtained for nearly all of the cycles.

An additional experiment was performed using the same primer, 37 C.8N.CG, sequencing bases 10, 11, 12 on T4 (except with emulsion beads instead of loaded beads, and showing only well-amplified, clonal beads). The signal on these beads was higher than the loaded beads. Without intending to be bound by theory, reasons for this include: a) more template on amplified beads; and (b) the switch to SEQUENASE™ from Klenow.

### EXAMPLE V

### Wobble Ligation Method

The following describes an embodiment of the invention referred to as “Wobble Ligation.” Several of the principles are identical or similar to Wobble Extension as previously described herein. These principles are distinguishable from FISSEQ and other sequencing methods, such as that described in Macevicz U.S. Pat. No. 5,750,341.

According to the Wobble Ligation embodiment described herein:

(a) At each step of the sequencing, a single base position in the unknown sequence is being queried.

(b) Which base is being queried is directly a function of the structure of the oligonucleotides used in the reaction.

(c) After each cycle of enzymatic treatment and imaging, these oligonucleotides are stripped from the DNA attached to the beads; the method is thus non-progressive, in that any given cycle is not dependent on the efficiency of previous cycles.

There are several differences between Wobble Extension and Wobble Ligation:

(a) Ligases, rather than polymerases, are used as the discriminatory enzyme,

(b) In Wobble Extension, a single primer is hybridized and extended; degenerate bases within the oligonucleotide primer are included to ‘reach’ a specific distance into the unknown sequence. In Wobble Ligation, a single primer is hybridized that is universal (the ‘anchor’ primer) and sits such that either its 5′ or 3′ end is immediately adjacent to the unknown sequence. The position to be queried is encoded in a pool of degenerate nonamers (9-mer) that are ligated to the anchor primer. However, anchor primers having one or several degenerate positions at the terminus to be ligated to can serve as substrates for ligation and so can be used to position the query even further into the unknown sequence.

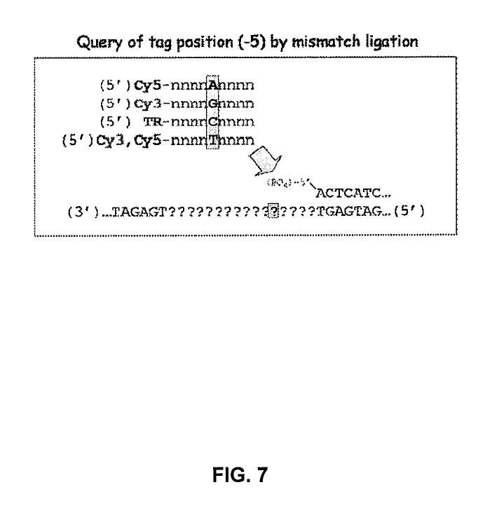

(c) The assays are always identical, in that the full pool of possible nonamers is being ligated to the anchor primer. What changes between the assays (and determines whether one is sequencing base 4 or base 7 in a particular cycle, for example), is the correlations between specific positions in the degenerate nonamer and fluorescent labels at its end. FIG. 7 depicts, for example, the querying of position (-4) relative to the anchor primer.

### EXAMPLE VI

### Ultra Low-Error PCR colonies

There is generally a high error rate for any pre-sequencing amplification method which starts from single templates and employs exponential amplification, including PCR, emlusion PCR, bead emulsion PCR, in situ polonies, digital PCR, bridge PCR, multiple displacement amplification (MDA) and the like. Such methods are described in C. P. Adams, S. J. Kron. (U.S. Pat. No. 5,641,658, Mosaic Technologies, Inc.; Whitehead Institute for Biomedical Research, USA, 1997); D. Dressman, H. Yan, G. Traverso, K. W. Kinzler, B. Vogelstein, Proc. Natl. Acad. Sci. USA, 100, 8817 (Jul. 22, 2003); D. S. Tawfik, A. D. Griffiths, Natl. Biotechnol., 16, 652 (Jul., 1998); F. J. Ghadessy, J. L. Ong, P. Holliger, Proc. Natl. Acad. Sci. USA, 98, 4552 (Apr. 10, 2001); M. Nakano et al., J. Biotechnol., 102, 117 (Apr. 24, 2003); R. D. Mitra, G. M. Church, Nucleic Acids Res 27, e34 (Dec 15, 1999); and F. B. Dean et al., Proc. Natl. Acad. Sci. USA, 99, 5261 (Apr. 16, 2002), each of which are hereby incorporated by reference.

Such error establishes an upper limit on the accuracy of any sequencing method which operates on material that is the product of the amplification. For example, during bead emulsion PCR, template is diluted to the point where 1 template molecule and 1 bead will be trapped in an emulsion compartment, and PCR will proceed from this single molecule resulting in many copies bound to the bead. An error arising early during the amplification will result in a bead having either a homogenous population of amplicons bearing the error, or a heterogenous population of amplicons, some bearing the error and some not. In either case, the accuracy of the sequence derived from such a bead will be low.

According to embodiments of the present invention, emulsion PCR will be started with multiple copies of a given template molecule in a compartment. Then, PCR will initiate from each copy independently, and the product bound to the bead in that compartment will be largely homogenous and error-free, even if errors arise early during amplification from 1 of the copies of the template.

To achieve this goal, two techniques are useful. The first is to clone the template desired to be sequenced into a plasmid, transform into bacteria or yeast, and perform emulsion PCR not with naked single-copy template DNA, but rather with individual cells, each of which includes multiple copies of the template. During PCR the cells will rupture and amplification will proceed from each copy of the plasmid present. Since multiple copies of the template were present, and since each was copied independently by the host cell's low-error replication machinery, the probability of obtaining a PCR-based error in a preponderance of amplicons is very low.

The second approach uses linear rolling circle amplification to prepare template molecules which are linear concatemers of independent copies of the original template. PCR then initiates from each site on the concatemer independently. The important constraint (regardless of the method used to get multiple copies of a template into an emulsion compartment or otherwise to initiate a spatially-clustered exponential amplification) is that the initial copies made of the original template are independent of each other and so the probability of two such copies bearing the same error is very low. With a linear rolling circle amplification, the original template (a circular molecule) is iterated over many times, such that all copies are copies of the original template (unlike PCR, which makes copies of copies).

### EXAMPLE VII

### Ligase-Driven DNA Molecular Ruler

Embodiments of the present invention are directed to methods to determine, with single-base resolution, the length of the unique region of a library molecule. To perform polony sequencing, a paired-tag genomic library is constructed where each library molecule is comprised of a unique region flanked by common primer sites. In order to generate a library where all inserts are short and of strictly defined length (which is important for signal homogeneity when using emulsion PCR to load the templates to sequencing beads), the type IIs restriction enzyme MmeI is used. MmeI cuts either 17 bp or 18 bp from its recognition sequence, and in the embodiment described here thus produces inserts of 17 bp or 18 bp at a ratio of about 50:50 with little to no sequence-dependence. Knowing the exact length of each insert is advantageous since sequencing methods described herein include the step of reading a certain number of bases from each side of the 17-18 bp tag. In order to generate a contiguous sequence from such reads, knowing the exact length of the insert would be beneficial.

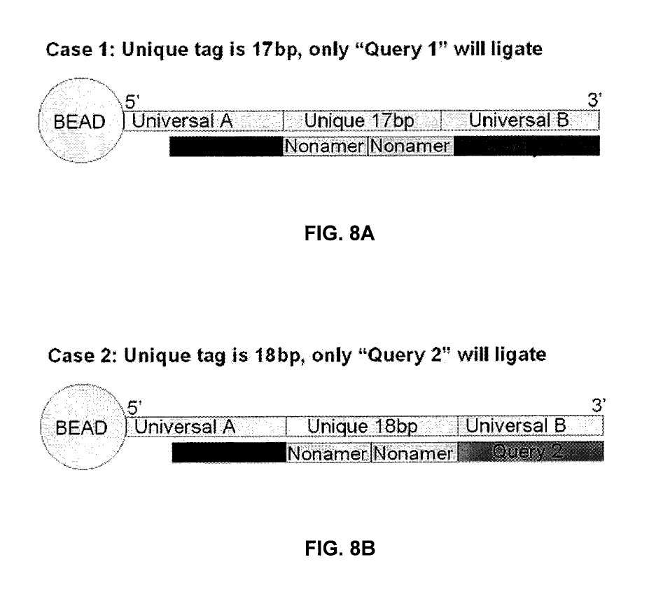

According to this embodiment a ligation-query scheme is used which relies on the specificity of the ligase reaction catalyzed by ampligase or some other ligase capable of yielding sufficient base paring specificity to first ‘walk’ across the insert with fully degenerate nonamers, and then query the identity of a base in the opposing universal primer sequence. An ‘anchor’ primer complementary to sequence in universal primer A can be first hybridized, then perform degenerate nonamer ligation to span the unique insert, and finally query the length of such insert with a pair of fluorescently-labeled query primers, where each possible length (17 or 18) is coded by a different fluorophore as depicted in FIGS. 8A and 8B.

### EXAMPLE VIII

An additional embodiment of the present invention is described in the following method.

1. Hybridize 5′-phosphorylated, deoxyuridine-containing anchor-primer to target sequence

3′-AGAGUCUACUCA-/5′Phos/
5′.....TCTCAGATGAGT???????????????...

2. Perform a base-query by ligating to this, with T4 DNA ligase, fully degenerate nonamers, where an internal base correlates with the identity of one of four fluorophores (four color nonamers) as illustrated in FIG. 7.

3. Collect data by four-color imaging or some other means.

4. To remove the primer:degenerate-sequence:fluorophore complex before beginning the next cycle, treat with both Endonuclease 8 and E. coli Uracil-DNA Glycosylase (“UDG”). The UDG will cleave the uracils in the anchor primer, leaving abasic sites that will be cleaved by Endonuclease 8, leaving short fragments with low Tm's that will melt off the immobilized DNA strands at ambient temperatures. Heat, chemical denaturants, or other chemically or enzymatically labile bonds in the anchor primer could also be used in place of deoxyuridines to remove the primer:degenerate-sequence:fluorophore complex.

This embodiment can be carried out in the 5′→3′ direction by using a degenerate nonamer population that is phosphorylated at the 5′ end (such that that end will ligate to the anchor primer), and the fluorophore resides on its 3′ end.

A kit including endonuclease 8 and UDG is commercially available from New England Biolabs under the tradename USER. A schematic of a sample UDG reaction is provided in the figure below.

[image]

### EXAMPLE IX

### Non-Progressive Cycling as Described in Example V

Certain polymerase- and ligase- driven cyclic sequencing methods are termed “progressive,” in that they interrogate the sequencing template by incorporating onto the end of a growing polynucleotide chain, digesting from the end of the template, or ligating to a growing oligonucleotide primer. See for example , Braslavsky, B. Hebert, E. Kartalov, S. R. Quake, Proc. Natl. Acad. Sci. USA, 100, 3960 (Apr. 1, 2003); R. D. Mitra, J. Shendure, J. Olejnik, O. Edyta Krzymanska, G. M. Church, Anal. Bioche ., 320, 55 (Sep. 1, 2003); M. Ronaghi, S. Karamohamed, B. Pettersson, M. Uhlen, P. Nyren, Anal. Bioche., 242, 84 (Nov. 1, 1996); S. C. C. Macevicz. (U.S. Pat. No. 5,750,341, Lynx Therapeutics, Inc., USA, 1998), and S. Brenner et al., Natl. Biotechnol., 18:630 (June 2000) each of which are hereby incorporated by reference. These “progressive” methods, however, are disadvantageous in that they exhibit amp licon dephasing, which results in decreased sequencing fidelity as the number of bases sequenced into the template increases.

The non-progressive cycling method of the present invention reduces, or in certain embodiments, eliminates, the adverse effects of amplicon dephasing in existing sequencing by synthesis methods (both polymerase- and ligase- driven) by removing the sequencing primer periodically (as often as after each base-position is interrogated). Thus, enzymatic and chemical inefficiencies and other errors do not accumulate as the sequencing run proceeds. Rather, each cycle is independent of previous inefficiencies or misincorporations (assuming the primer is removed after each sequencing cycle). The non-progressive cycling method of the present invention has the added advantage of allowing one to know, with reasonably certainty, which position in the template is being interrogated. This advantageously allows one to resolve homopolymers since the interrogation event has been de-coupled from the positioning event. Furthermore, it allows one to sequence a template out-of-order, rather than requiring one to sequentially query positions 5′ to 3′ or 3′ to 5′.

According to the non-progressive cycling method of the present invention, the primer can be removed in a number of ways. Heat can be used to melt the primer off the template. Alkali can be used to chemically denature the primer from the template. Numerous other chemical denaturants can be used, which include: methanol, ethanol, isopropanol, n-propanol, allyl alcohol, sec-butyl alcohol, tert-butyl alcohol, isobutyl alcohol, n-butyl alcohol, tert-amyl alcohol, ethylene glycol, glycerol, dithioglycerol, propylene glycol, cyclohexyl alcohol, benzyl alcohol, inositol, phenol, p-methoxyphenol, aniline, pyridine, purine, 1,4-dioxane, gamma-butyrolactone, 3-amino triazole, formamide, N-ethyl formamide, N-N-dimethylformamide, acetamide, N-ethyl acetamide, N-N-dimethyl acetamide, propionamide, butyramide, hexamide, glycolamide, thioacetamide, delta-valerolactam, urethan, N-methyl urethan, N-propylurethan, cyanoguanidine, sulfamide, glycine, acetonitrile, urea, Tween 40, Triton X-100, sodium trichloroacetate, sodium perchlorate, lithium bromide, cesium chloride, lithium chloride, potassium thiocyanate, sodium trifluoroacetate, sodium dodecyl sulfate, salicylate, dimethylsulfoxide, dioxane, and the like. Suitable denaturation methods are described in L. Levine, J. A. Gordon, W. P. Jencks, Biochem. 2:168 (January 1963); and J. Shendure et al., Science (published online Aug. 4, 2005).

Chemically-labile linkages, such as phosphorothioate with heavy-metal ion cleavage treatment as described in M. Mag, S. Luking, J. W. Engels, Nucleic Acids Res., 19:1437 (Apr. 11, 1991) can be included in the primer to allow it to be fragmented into many pieces, each of which has a Tm low enough to cause the primer:query complex to denature from the template. Primers can be made enzymatically-labile by the inclusion of ribonucleotides or ribonucleotide stretches (susceptible to cleavage by RNase H or alkali) or the inclusion of deoxyuridines (subject to cleavage by a mixture of uracil DNA glycosylase and endonuclease VIII) or abasic sites (subject to cleavage by endonuclease VIII). The primer can also be removed enzymatically by the use of a suitable exonuclease.

Non-Progressive Sequencing By Ligation Using Deoxyuridine Stripping

According to one aspect of the present invention, the following steps were carried out cyclically to interrogate each base of the template sequentially. An ‘anchor primer’ was hybridized complementary to common library sequence. A pool of fluorescently-labeled ‘query primers’ specific to one tag-position was then ligated to the template. Imaging was then used to determine which primer pool ligated to which bead. The anchor::query primer complex was then stripped. The process was then repeated.

Anchor primers used had the following sequences (U=deoxyuridine):

| | |
|---|---|
| T30UIA | 5′-GGGCCGUACGUCCAACT-3′ |
| T30UIB | 5′-CGCCUUGGCCUCCGACT-3′ |
| PR1U10N | 5′-CCCGGGUUCCUCAUUCUCT-3′ |
| LIGFIXDD | 5′-Phos/AUCACCGACUGCCCA-3′ |
| LIGFIXD2T30A | 5′-Phos/AGUUGGAGGUACGGC-3′ |
| LIGFIXD2T30B | 5′-Phos/AGUCGGAGGCCAAGC-3′ |

Query primers used were nonamers which were degenerate at all positions excepy the query position. At the query position, only one base was present for a given fluorophore. For example, the pool of probes used to query position five was composed of the following four label-subpools:

| | |
|---|---|
| Cy54NA | 5′-Phos/NNNNANNNN/Cy5--3′ |
| Cy34NG | 5′-Phos/NNNNGNNNN/Cy3-3′ |
| TexasRed4NC | 5′-Phos/NNNNCNNNN/TR-3′ |
| FRET4NT | 5′-Phos/NNNNTNNNN/FRET-3′ |

Anchor primers were hybridized in a flowcell (lOOuM primer in 6× SSPE) for 5 minutes at 56 C, then cooled to 42 C and held for 2 minutes. Excess primer was then washed out at room temperature with Wash IE (10 mM Tris-HCl pH 7.5, 50 mM KCl, 2 mM EDTA pH 8.0, 0.01% Triton X-100) for 2 minutes.

Query primers were ligated in the flowcell (8 uM query primer mix (2 uM each subpool), 6000U T4 DNA ligase (NEB), 1×T4 DNA ligase buffer (NEB)) at 35 C and held for 30 minutes. At the end of the reaction, excess query primer was washed out at room temperature with Wash 1 E for 5 minutes.

Four-color imaging was performed on an epifluorescence microscope with filters appropriate to the fluorophores attached to the nonamers.

Anchor::query primer complex was stripped with USER (NEB), a combination of uracil DNA glycosylase and endonuclease VIII. To perform the stripping reaction, the following protocol was executed in the flowcell:
Incubate 150 uL stripping mix (3 ul USER (NEB), 150 ul TE) for 5 minutes at 37 C
Raise temperature to 56 C and hold 1 minute
Wash for Iminute with Wash IE; temperature gradually decreases
Incubate 150 ul fresh stripping mix for 5 minutes at 37 C
Wash for 5 minutes with Wash IE; temperature gradually decreases

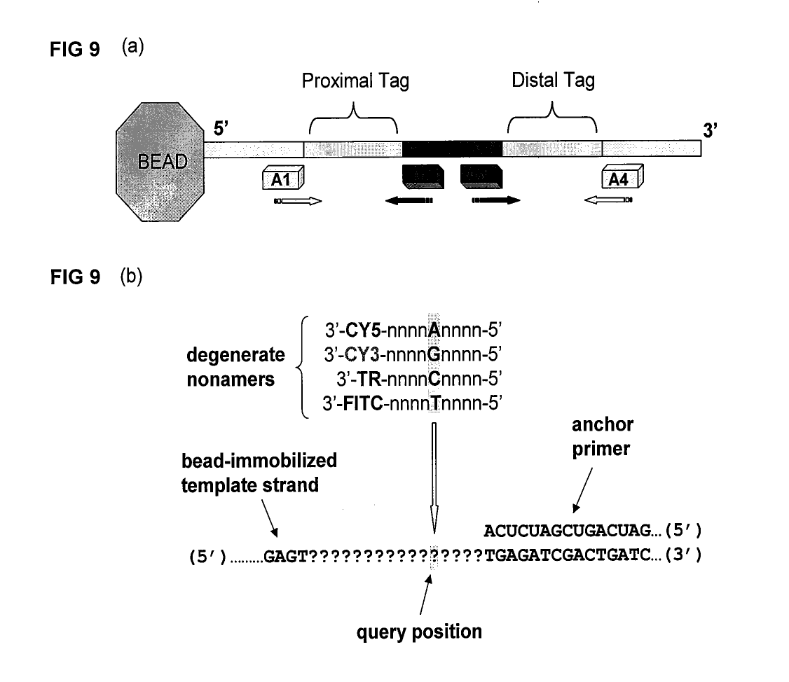

With reference to FIG. 9A, the cycles consist of the following four steps: (a) hybridization of one of four anchor primer, (b) ligation of fluorescent, degenerate nonamers, (c) four color imaging on epifluorescence microscope, (d) stripping of the anchor primer:nonamer complexes prior to beginning the next cycle. The anchor primers are each designed to be complementary to universal sequence immediately 5′ or 3′ to one of the two tags. A1, A2, A3 and A4 indicate the four locations to which anchor primers are targeted relative to the amplicon. Arrows indicate the direction sequenced into the tag from each anchor primer. From anchor primers Al and A3, 7 bases are sequenced into each tag, and from anchor primers A2 and A4, 6 bases are sequenced into each tag. Thus, 13 bp per tag are obtained, and 26 bp per amplicon, with 4 to 5 bp gaps within each tag sequence. With reference to FIG. 9B, each cycle involves performing a ligation reaction with T4 DNA ligase and a fully degenerate population of nonamers. The nonamer molecules are individually labeled with one of four fluorophores (e.g., Texas Red, Cy5, Cy3, FITC). Depending on which position that a given cycle is aiming to interrogate, the nonamers are structured differently. Specifically, a single position within each nonamer is correlated with the identity of the fluorophore with which it is labeled. Additionally, the fluorphore molecule is attached at the opposite end of the nonamer relative to the end targeted to the ligation junction. For example, in FIG. 9B, the anchor primer is hybridized such that its 3′ end is adjacent to the genomic tag. To query a position five bases in to the tag sequence, the four-color population of nonamersis used.

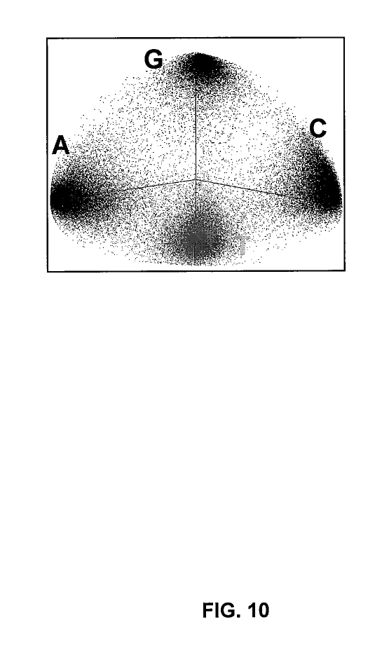

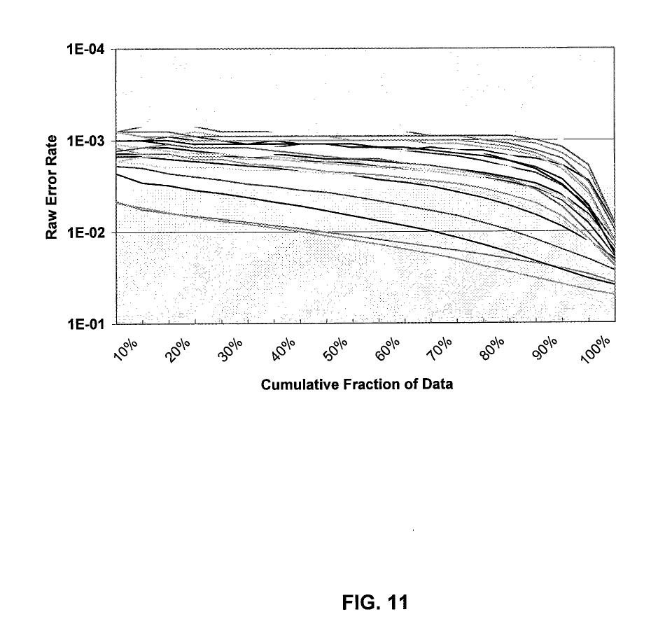

Referring to FIG. 10, four-color data from each cycle can be visualized in tetrahedral space, where each point represents a single bead, and the four clusters correspond to the four possible base calls. FIG. 11 shows data from a single cycle of non-progressive sequencing by ligation, and in particular is the sequencing data from position (-1) of the proximal tag of a complex E. coli derived library. FIG. 11 shows variation in accuracy over each of 26 cycles of non-progressive sequencing by ligation in a single experiment resequencing an E. coli genome. Cumulative distribution of raw error as a function of rank-ordered quality, with each of 26 sequencing-by-ligation cycles in a single sequencing experiment is treated as an independent data-set. The x-axis indicates percentile bins of beads, sorted on the basis of a confidence metric. The y-axis (log scale) indicates the raw base-calling accuracy of each cumulative bin.

### REFERENCES

Housby J N, Southern E M., “Thermus scotoductus and Rhodothermus marinus DNA ligases have higher ligation efficiencies than thermus thermophilus DNA ligase,” Anal Biochem., 2002 Mar. 1; 302(1):88-94.

Housby J N, Thorbjarnardottir S H, Jonsson Z O, Southern E M., “Optimised ligation of oligonucleotides by thermal ligases: comparison of Thermus scotoductus and Rhodothermus marinus DNA ligases to other thermophilic ligases,” Nucleic Acids Res., 2000 Feb. 1; 28(3):E10.

Housby J N, Southern E M., “Fidelity of DNA ligation: a novel experimental approach based on the polymerisation of libraries of oligonucleotides,” Nucleic Acids Res., 1998 Sep. 15; 26(18):4259-4266.

Pritchard C E, Southern E M., “Effects of base mismatches on joining of short oligodeoxynucleotides by DNA ligases,” Nucleic Acids Res., 1997 Sep. 1; 25(17):3403-3407.

What is claimed is:

1. A method described above for DNA sequencing, useful for sequencing homopolymeric regions of DNA.

2. A method of sequencing a target nucleic acid comprising:
a. providing a sequencing primer, wherein the sequencing primer has at least one anchor sequence and a universal base;
b. hybridizing the sequencing primer to a target nucleic acid; and
c. extending the sequencing primer.

3. A method of sequencing a target nucleic acid comprising:
a. providing a sequencing primer, wherein the sequencing primer has at least one anchor sequence and a degenerate base;
b. hybridizing the sequencing primer to a target nucleic acid; and
c. extending the sequencing primer.

4. A method of sequencing a target nucleic acid comprising:
a. providing a sequencing primer, wherein the sequencing primer has at least one anchor sequence and a natural base;
b. hybridizing the sequencing primer to a target nucleic acid; and
c. extending the sequencing primer.

5. A method for sequencing a target nucleic acid comprising:
(a) hybridization of one of several anchor primers to a common sequence adjacent to an unknown sequence,
(b) ligation of fluorescently labeled, degenerate oligonucleotides to the anchor primer, such that identity of the fluorophore is informative of the identity of one or more positions within the degenerate oligonucleotide,
(c) imaging to determine primer ligation,
(d) stripping of the anchor primer:degenerate oligonucleotide complexes, and
(e) repeating steps (a)-(d) one or more times.


SEQUENCE LISTING (47 sequences)

SEQ ID NO: 1 - DNA, 13 bp, artificial sequence
  primer
cctcattctctca

SEQ ID NO: 2 - DNA, 13 bp, artificial sequence
  primer
cctcattctctgt

SEQ ID NO: 3 - DNA, 13 bp, artificial sequence
  primer
cctcattctctat

SEQ ID NO: 4 - DNA, 13 bp, artificial sequence
  primer
cctcattctctag

SEQ ID NO: 5 - DNA, 15 bp, artificial sequence
  primer
  misc_feature (12)..(13): wherein n is g, a, t or c
cctcattctctnnca

SEQ ID NO: 6 - DNA, 15 bp, artificial sequence
  primer
  misc_feature (12)..(13): wherein n is g, a, t or c
cctcattctctnngt

SEQ ID NO: 7 - DNA, 15 bp, artificial sequence
  primer
  misc_feature (12)..(13): wherein n is g, a, t or c
cctcattctctnntg

SEQ ID NO: 8 - DNA, 15 bp, artificial sequence
  primer
  misc_feature (12)..(13): wherein n is g, a ,t or c
cctcattctctnngc

SEQ ID NO: 9 - DNA, 17 bp, artificial sequence
  primer
  misc_feature (12)..(15): wherein n is g, a, t or c
cctcattctctnnnnca

SEQ ID NO: 10 - DNA, 17 bp, artificial sequence
  primer
  misc_feature (12)..(15): wherein n is g, a, t or c
cctcattctctnnnngt

SEQ ID NO: 11 - DNA, 17 bp, artificial sequence
  primer
  misc_feature (12)..(15): wherein n is g, a, t or c
cctcattctctnnnnct

SEQ ID NO: 12 - DNA, 17 bp, artificial sequence
  primer
  misc_feature (12)..(15): wherein n is g, a, t or c
cctcattctctnnnnga

SEQ ID NO: 13 - DNA, 17 bp, artificial sequence
  primer
  misc_feature (12)..(15): wherein n is g, a, t or c
cctcattctctnnnncg

SEQ ID NO: 14 - DNA, 19 bp, artificial sequence
  primer
  misc_feature (12)..(17): wherein n is g, a, t or c
cctcattctctnnnnnnca

SEQ ID NO: 15 - DNA, 19 bp, artificial sequence
  primer
  misc_feature (12)..(17): wherein n is g, a, t or c
cctcattctctnnnnnngt

SEQ ID NO: 16 - DNA, 19 bp, artificial sequence
  primer
  misc_feature (12)..(17): wherein n is g, a, t or c
cctcattctctnnnnnnaa

SEQ ID NO: 17 - DNA, 19 bp, artificial sequence
  primer
  misc_feature (12)..(17): wherein n is g, a, t or c
cctcattctctnnnnnnga

SEQ ID NO: 18 - DNA, 21 bp, artificial sequence
  primer
  misc_feature (12)..(19): wherein n is g, a, t or c
cctcattctctnnnnnnnnca

SEQ ID NO: 19 - DNA, 21 bp, artificial sequence
  primer
  misc_feature (12)..(19): wherein n is g, a, t or c
cctcattctctnnnnnnnngt

SEQ ID NO: 20 - DNA, 21 bp, artificial sequence
  primer
  misc_feature (12)..(19): wherein n is g, a, t or c
cctcattctctnnnnnnnncg

SEQ ID NO: 21 - DNA, 21 bp, artificial sequence
  primer
  misc_feature (12)..(19): wherein n is g, a, t or c
cctcattctctnnnnnnnngc

SEQ ID NO: 22 - DNA, 19 bp, artificial sequence
  primer
  misc_feature (12)..(17): wherein n is g, a, t or c
cctcattctctnnnnnngt

SEQ ID NO: 23 - DNA, 19 bp, artificial sequence
  primer
  misc_feature (12)..(17): wherein n is g, a, t or c
cctcattctctnnnnnnga

SEQ ID NO: 24 - DNA, 21 bp, artificial sequence
  primer
  misc_feature (10)..(19): wherein n is g, a, t or c
tcattctctnnnnnnnnnnac

SEQ ID NO: 25 - DNA, 25 bp, artificial sequence
  primer
  misc_feature (12)..(23): wherein n is g, a, t or c
cctcattctctnnnnnnnnnnnncg

SEQ ID NO: 26 - DNA, 27 bp, artificial sequence
  primer
  misc_feature (12)..(25): wherein n is g, a, t or c
cctcattctctnnnnnnnnnnnnnnat

SEQ ID NO: 27 - DNA, 29 bp, artificial sequence
  primer
  misc_feature (12)..(27): wherein n is g, a, t or c
cctcattctctnnnnnnnnnnnnnnnncc

SEQ ID NO: 28 - DNA, 31 bp, artificial sequence
  primer
  misc_feature (12)..(29): wherein n is g, a, t or c
cctcattctctnnnnnnnnnnnnnnnnnncc

SEQ ID NO: 29 - DNA, 26 bp, artificial sequence
  template
cacacacacacacacactccaccact

SEQ ID NO: 30 - DNA, 26 bp, artificial sequence
  template
gtgtgtgtgtgtgtgtccaccactct

SEQ ID NO: 31 - DNA, 26 bp, artificial sequence
  template
agtgctcacacacgtgatccaccact

SEQ ID NO: 32 - DNA, 26 bp, artificial sequence
  template
cagccgaacgaccgatccaccactct

SEQ ID NO: 33 - DNA, 26 bp, artificial sequence
  template
atgtgagagctgtcgtccaccactct

SEQ ID NO: 34 - RNA, 15 nt, artificial sequence
  primer
gaucagucgaucuca

SEQ ID NO: 35 - DNA, 15 bp, artificial sequence
  primer
tgagatcgactgatc

SEQ ID NO: 36 - RNA, 12 nt, artificial sequence
  primer
  misc_feature (1)..(1): 5′ phosphorylation
acucaucugaga

SEQ ID NO: 37 - DNA, 12 bp, artificial sequence
  primer
tctcagatgagt

SEQ ID NO: 38 - DNA, 17 bp, artificial sequence
  primer
  misc_feature (7)..(7): n is deoxyuridine
  misc_feature (11)..(11): n is deoxyuridine
gggccgnacgnccaact

SEQ ID NO: 39 - DNA, 17 bp, artificial sequence
  primer
  misc_feature (5)..(6): n is deoxyuridine
  misc_feature (11)..(11): n is deoxyuridine
cgccnnggccnccgact

SEQ ID NO: 40 - DNA, 19 bp, artificial sequence
  primer
  misc_feature (7)..(8): n is deoxyuridine
  misc_feature (11)..(11): n is deoxyuridine
  misc_feature (14)..(15): n is deoxyuridine
  misc_feature (17)..(17): n is deoxyuridine
cccgggnnccncanncnct

SEQ ID NO: 41 - DNA, 15 bp, artificial sequence
  primer
  misc_feature (1)..(1): 5′ phosphorylation
  misc_feature (2)..(2): n is deoxyuridine
  misc_feature (10)..(10): n is deoxyuridine
ancaccgacngccca

SEQ ID NO: 42 - DNA, 15 bp, artificial sequence
  primer
  misc_feature (1)..(1): 5′ phosphorylation
  misc_feature (3)..(4): n is deoxyuridine
  misc_feature (10)..(10): n is deoxyuridine
agnnggaggnacggc

SEQ ID NO: 43 - DNA, 15 bp, artificial sequence
  primer
  misc_feature (1)..(1): 5′ phosphorylation
  misc_feature (3)..(3): n is deoxyuridine
agncggaggccaagc

SEQ ID NO: 44 - DNA, 9 bp, artificial sequence
  primer
  misc_feature (1)..(1): 5′ phosphorylation
  misc_feature (1)..(4): wherein n is g, a, t or c
  misc_feature (6)..(9): wherein n is g, a, t or c
  misc_feature (9)..(9): 3′ Cy5 label
nnnnannnn

SEQ ID NO: 45 - DNA, 9 bp, artificial sequence
  primer
  misc_feature (1)..(1): 5′ phosphorylation
  misc_feature (1)..(4): wherein n is g, a, t or c
  misc_feature (6)..(9): wherein n is g, a, t or c
  misc_feature (9)..(9): 3′ Cy3 label
nnnngnnnn

SEQ ID NO: 46 - DNA, 9 bp, artificial sequence
  primer
  misc_feature (1)..(1): 5′ phosphorylation
  misc_feature (1)..(4): wherein n is g, a, t or c
  misc_feature (6)..(9): wherein n is g, a, t or c
  misc_feature (9)..(9): 3′ Texas Red label
nnnncnnnn

SEQ ID NO: 47 - DNA, 9 bp, artificial sequence
  primer
  misc_feature (1)..(1): 5′ phosphorylation
  misc_feature (1)..(4): wherein n is g, a, t or c
  misc_feature (6)..(9): wherein n is g, a, t or c
  misc_feature (9)..(9): 3′ fluorescent energy transfer label
nnnntnnnn